(19) United States
(12) Patent Application Publication
(10) Pub. No.: US 2023/0109454 A1
(43) Pub. Date: Apr. 6, 2023

(54) ABDOMINAL HEMORRHAGE CONTROL DEVICE AND METHOD

(71) Applicant: THE REGENTS OF THE UNIVERSITY OF MICHIGAN, Ann Arbor, MI (US)

(72) Inventors: Kevin Ward, Glen Allen, VA (US); Jeffrey Stephen Plott, Algonac, MI (US); Mohamad Hakam Tiba, Ann Arbor, MI (US); Brendan McCracken, Ypsilanti, MI (US)

(21) Appl. No.: 17/798,592

(22) PCT Filed: Feb. 12, 2021
(86) PCT No.: PCT/US2021/017889

§ 371 Date: Aug. 10, 2022

Related U.S. Application Data
(60) Provisional application No. 62/976,809, filed on Feb. 14, 2020.

(51) Int. Cl. A61B 17/12; A61B 17/34; A61M 1/00

(52) U.S. Cl. CPC A61B 17/12136; A61B 17/3415; A61B 17/12099; A61M 1/84; A61B 2017/00022; A61B 2017/12004

(57) ABSTRACT

A hemorrhage control device includes a trocar having an opening at a distal end. An expandable balloon is disposed within the trocar, the expandable balloon being collapsed within the trocar in a stored condition, and the expandable balloon being movable out of the trocar, through the opening, into a deployed condition. An inflation tube is fluidly connected to an interior of the expandable balloon. A source of pressurized fluid is connected to the inflation tube, pressurized fluid being introduced into the inflation tube to pressurize and inflate the expandable balloon in the deployed condition.

The present disclosure relates to hemorrhage control devices and methods and more particularly to abdominal hemorrhage control devices methods.

### BACKGROUND

Hemorrhagic shock is a leading cause of death from trauma. Many times there are delays in reaching hospitals that are qualified to take care of the complex injuries of such individuals. Many patients who die of trauma, die from multi-system involvement. Multi-system involvement may include head injury along with injuries to organs of the thoracic and abdominal cavities. Uncontrolled hemorrhage leading to hypovolemic shock is a leading cause of death from trauma especially from blunt and penetrating trauma of the abdomen and pelvis. When head trauma occurs concomitantly with thoracic and abdominal hemorrhage, the brain becomes hypoperfused and, thus, becomes at greater risk for secondary injury. Currently, in the pre-hospital and emergency department setting, there are limited means to control exsanguinating hemorrhage below the diaphragm while maintaining myocardial and cerebral blood flow. Definitive control of hemorrhage is performed at surgery but this may be delayed and may not occur within the golden hour (time from injury to definitive treatment/repair) where the best opportunity lies in salvaging the patient. Survival with improved neurologic outcome might be enhanced if means were available to slow or stop ongoing hemorrhage (especially below the diaphragm) while maintaining adequate perfusion to the heart and brain until definitive treatment of the hemorrhage is available. This would be especially true of trauma victims whose transport to appropriate medical facilities would be prolonged.

Non-compressible torso hemorrhage (NCTH) is one type of a major hemorrhage surgical emergency that is responsible for approximately 70% of military and 10% of civilian potentially survivable hemorrhage deaths. NCTH may be defined as a high-grade injury present in one or more of the following anatomic domains: pulmonary, solid abdominal organ, major vascular or pelvic trauma; plus hemodynamic instability or the need for immediate hemorrhage control. Most NCTH casualties in the theater of battle die in the prehospital environment, before surgical intervention can occur, and where there are for the most part no viable interventions available. Additionally, the National Trauma Data Bank estimated that greater than 20,000 U.S. civilians suffered from a non-compressible torso injury involving hemorrhage between 2007- 2009, of which 45% were fatal.

Rapid surgical management is the mainstay treatment of NCTH. In this regard, early pre-surgical management of NCTH poses a significant challenge. Current endovascular techniques such as the resuscitative endovascular balloon occlusion of the aorta (REBOA) are showing promise as a management strategy to control NCTH in hospital emergency departments and trauma center settings by implementing a complete or partial obstruction of blood flow to the affected area. However, REBOA requires femoral artery access which might require surgical or ultrasound guided placement making it difficult to implement in most non-trauma centers and in the out of hospital environment. There are many implementation barriers for timely and early application of REBOA and deployment for point of injury care may pose logistical challenges.

New emerging approaches are being developed and tested to control NCTH at the point of injury or during transport. One such method involves in-situ forming polymeric foams, such as ResQFoam™ (Arsenal Medical, Inc. Waltham, MA). In-situ forming polymeric foams are not currently approved by the FDA for clinical use. In-situ forming polymeric foams are self-expanding polyurethane foams designed to be injected into the abdominal cavity to stanch hemorrhage by covering and compressing bleeding surfaces. While such foams have been shown to improve survival in a large animal lethal, closed-cavity, hepato-portal injury models, the field feasibility of these foams remains under-established. Furthermore, the expansion of these foams cannot be controlled or titrated after injection, and has been associated with complications such as bowel injury and possibly abdominal compartment syndrome. In addition, patients receiving these foams require surgery to remove the foam material even if nonsurgical control of bleeding could be accomplished using techniques such as interventional radiology.

Other methods of slowing or stopping hemorrhage include the use of a pneumatic anti-shock garment (PASG). Use of the PASG has met with varying degrees of success depending on the location of injury. This garment is placed on the legs and abdomen and is then inflated. Hemorrhage in the abdominal cavity, as well as the lower extremities, is controlled through tamponade while systemic blood pressure is raised partially through autotransfusion and by raising peripheral vascular resistance. Use of the PASG can sometimes be cumbersome and does not uniformly control hemorrhage or raise blood pressure. In addition, persons with concomitant penetrating thoracic injuries may hemorrhage more when the device is applied. The device may also raise intracranial pressure, which might detrimentally alter cerebral blood flow resulting in neurologic injury. As a result, the PASG is no longer recommended for use for abdominal hemorrhage control.

Other more drastic methods of controlling abdominal bleeding prior to surgery include the use of thoracotomy to cross-clamp the thoracic aorta and the use of balloon catheters placed into the aorta from the femoral arteries to a point above the celiac-aortic axis. These techniques have met with varying degrees of success, require a high degree of skill, and cannot be performed in hospitals not equipped to care for trauma patients or by paramedical care personnel.

Deliberately keeping hemorrhaging trauma victims in a hypotensive state is currently being examined as a means to improve survival. This is done based on the premise that overall hemorrhage (especially abdominal hemorrhage) is reduced if mean arterial pressure is kept low by not aggressively volume-repleting the victim prior to surgery. Unfortunately, this may be dangerous for trauma victims with concomitant head injury or myocardial dysfunction.

An important cause of hemorrhagic shock not caused by trauma includes rupture of abdominal aortic aneurysms. These can occur suddenly and without warning. Control of bleeding even at surgery can be difficult. Temporary measures discussed above for hemorrhage secondary to trauma have been tried for hemorrhage secondary to aneurysm rupture. The same difficulties apply. Survival might be enhanced if hemorrhage could be controlled earlier while maintaining perfusion to the heart and brain.

U.S. Pat. No. 5,531,776, and U.S. Pat. Publication No. 2002/0016608, the disclosures of which are hereby incorporated herein by reference, each disclose non-invasive techniques for partially or completely occluding the descending aorta. While these techniques have been at least somewhat successful at reducing hemorrhagic shock, these methods and devices have not gained widespread acceptance due to some difficulty in advancing and properly placing the devices in a patient. Additionally, these methods require particular orientations of the devices for correct operation. Finding and maintaining the precise orientation can be difficult.

Finally, in some cases, external pressure may be necessary to stop internal or external hemorrhages. In some areas of the body, for example in the abdomen, external pressure is normally applied by hand. Hand pressure can be cumbersome and in the case where there is only one person to help, hand pressure removes one hand available to accomplish other tasks. Tourniquets may be used in some cases, but tourniquets can result in limb complications and cannot be used on the trunk of the body.

The dire outcomes for patients suffering from NCTH and the limitations of current management methods highlight the urgency to develop new modalities that rapidly stop NCTH hemorrhage in the prehospital or austere environments, as well as medical treatment facilities that are not trauma centers.

### SUMMARY

In a first example, a hemorrhage control device includes a trocar having an opening at a distal end. An expandable balloon is disposed within the trocar, the expandable balloon being collapsed within the trocar in a stored condition, and the expandable balloon being movable out of the trocar, through the opening, into a deployed condition. An inflation tube is fluidly connected to an interior of the expandable balloon. A source of pressurized fluid is connected to the inflation tube, pressurized fluid being introduced into the inflation tube to pressurize and inflate the expandable balloon in the deployed condition.

In a second example, a hemorrhage control device includes a trocar having an opening at a distal end. An expandable balloon is disposed within the trocar, the expandable balloon being collapsed within the trocar in a stored condition, and the expandable balloon being movable out of the trocar, through the opening, into a deployed condition. An inflation tube is fluidly connected to an interior of the expandable balloon. An inflation baffle extends from a distal end of the inflation tube at least partially into the expandable balloon, the inflation baffle including a plurality of fluid openings that fluidly connect the inflation tube to the interior of the expandable balloon.

In a third example, a method of occluding a non-compressible abdominal hemorrhage includes introducing an expandable balloon into a peritoneal cavity through a trocar. The expandable balloon is moved out of the trocar; and inflated in the peritoneal cavity of a patient. Pressure is applied to a hemorrhage site by the expandable balloon.

Any of the first, second, and third examples may include any one or more of the following optional forms.

In one optional form, the source of pressurized fluid comprises an inflation pump.

In another optional form, the expandable balloon is capable of containing internal pressures from about 0 mm hg to about 200 mm hg.

In yet another optional form, the expandable balloon has an external diameter of between about 2 cm and about 60 cm when internal pressure of the expandable balloon is at an operating pressure.

In yet another optional form, the trocar has a first cross-sectional diameter and the expandable balloon has a second cross-sectional diameter, the second cross-sectional diameter being measured when the expandable balloon is inflated to an operating pressure, and the second cross-sectional diameter capable of being greater than 5 times larger than the first cross-sectional diameter.

In yet another optional form, an inflation baffle is located within the expandable balloon, the inflation baffle being fluidly connected to the inflation tube. In some embodiments, the inflation baffle may be integrally formed with, and an extension of, the inflation tube.

In yet another optional form, the inflation baffle includes a plurality of fluid openings distributed along a length of the inflation baffle.

In yet another optional form, the plurality of fluid openings includes at least a first fluid opening proximate to a distal tip of the inflation baffle and a second fluid opening between the distal tip of the inflation baffle and the distal end of the inflation tube.

In yet another optional form, at least two fluid openings in the plurality of fluid openings are separated from one another by at least 5 mm.

In yet another optional form, at least two openings in the plurality of fluid openings are not axially aligned with one another.

In yet another optional form, a length of the inflation baffle is greater than 20%, preferably greater than 50%, and more preferably greater than 90%, of a diameter of the expandable balloon, when the expandable balloon is inflated to an operating diameter. Similarly, the length of the inflation baffle should extend across greater than 20%, preferably greater than 50%, and more preferably greater than 90% of the uninflated length (or diameter) of the expandable balloon.

In yet another optional form, the expandable balloon has an outer wall comprising a flexible material having a shore hardness of between about 70 A and 80 D and a wall thickness of between about 0.001 and 0.003 in.

In yet another optional form, the expandable balloon comprises a thermoplastic elastomer.

In yet another optional form, a distal end of the trocar comprises a deformable tip.

In yet another optional form, a distal end of the trocar comprises a curved tip.

In yet another optional form, a balloon plunger is slidably disposed within the trocar, the balloon plunger being capable of pushing the expandable balloon out of the trocar, through the opening.

In yet another optional form, a rigid external support member is included.

In yet another optional form, the source of pressurized fluid is a source of heated fluid or a source of cooled fluid.

In yet another optional form, a physiological sensor is connected to the expandable balloon or to the inflation tube.

In yet another optional form, the physiological sensor is one of a pressure sensor, a heart rate monitor, a photoplethysmograph, a pulse oximeter, and a thermometer.

In yet another optional form, applying pressure to the hemorrhage site includes applying indirect pressure by moving patient intra-abdominal tissues to occlude the hemorrhage site.

In yet another optional form, the indirect pressure is applied to the hemorrhage site by one of a patient’s intestines, stomach, omentum, liver and/or spleen, which is moved by the expandable balloon when the expandable balloon is inflated.

In yet another optional form, the trocar is removed before inflating the expandable balloon.

In yet another optional form, inflating the expandable balloon includes inflating the expandable balloon to an internal pressure of at least 20 mm hg.

In yet another optional form, internal balloon pressure is adjusted based on continued bleeding or blood pressure readings.

In yet another optional form, supplemental pressure is applied external to the peritoneal cavity.

In yet another optional form, external pressure is applied in conjunction with a back plate connected to an external abdominal plate allowing for external pressure to be adjusted to achieve hemostasis.

In yet another optional form, external pressure is applied by one of circumferential inflatable wrap or a belt.

In yet another optional form, fluid is extracted from the peritoneal cavity through a lumen separate from an inflation tube or a lumen within the inflation tube.

In yet another optional form, hemostatic, antibacterial, or resuscitative adjuncts are introduced into the peritoneal cavity through a lumen separate from an inflation tube or a lumen within the inflation tube.

In yet another optional form, one of heated fluid or cooled fluid is introduced into the expandable balloon.

In yet another optional form, an internal pressure of the expandable balloon is adjusted based on physiological measurements.

In yet another optional form, the physiological measurements are taken by sensors connected to the expandable balloon and/or by sensors connected to a patient’s body external to the peritoneal cavity.

In yet another optional form, the expandable balloon is wrapped around an inflation baffle before introducing an expandable balloon into a peritoneal cavity through the trocar.

In yet another optional form, the expandable balloon is folded into a plurality of pleats around an inflation baffle before introducing the expandable balloon into a peritoneal cavity through the trocar.

### BRIEF DESCRIPTION OF THE DRAWINGS

FIG. 1 is a schematic view of a hemorrhage control device deployed in an abdominal cavity of a patient.

FIG. 2 is a plan view of a first embodiment of the hemorrhage control device of FIG. 1.

FIG. 3 is a plan view of a second embodiment of the hemorrhage control device of FIG. 1.

FIG. 4 is a plan view of the hemorrhage control device of FIG. 3, with an expandable balloon in a deployed and inflated state.

FIG. 5A is a side cross-sectional view of the expandable balloon of FIG. 4, with an inflation baffle spanning part of a diameter of the expandable balloon.

FIG. 5B is a side cross-sectional view of the expandable balloon of FIG. 4, with an inflation baffle spanning the entire diameter of the expandable balloon.

FIG. 5C is a perspective view of the expandable balloon of FIG. 4, wrapped around the inflation baffle and installed in a trocar.

FIG. 5D is a perspective view of the expandable balloon of FIG. 5C, partially deployed from the trocar, and partially wrapped around the inflation baffle before inflation.

FIG. 6 is a side perspective view of the inflation baffle of FIG. 5A.

FIG. 7A is a perspective view of an external pressure device that may be used with the hemorrhage control device.

FIG. 7B is a side view of the hemorrhage control device deployed in a body cavity with external pressure being applied by a human hand.

FIG. 8 is another view of the external pressure device of FIG. 7A.

FIG. 9 is a graph of experimental data illustrating the effectiveness of the hemorrhage control device for full aortic occlusion.

FIG. 10 is a graph of experimental data illustrating the effectiveness of the hemorrhage control device for partial aortic occlusion.

FIG. 11 is a graph of survival probability of test subjects with and without use of the hemorrhage control device.

### DETAILED DESCRIPTION

A hemorrhage control device, as described herein, may be used in conjunction with, or without, an external pressure device. The disclosed hemorrhage control device and external pressure device may be used together to slow or stop bleeding in the lower part of the body below the diaphragm. However, each device may also be used separately to stop other types of bleeding. The disclosed hemorrhage control device may advantageously be used to stop abdominal and/or pelvic bleeding with direct or indirect pressure. Direct pressure, as defined herein, means that an expandable balloon of the device presses directly against a hemorrhage location. Indirect pressure, as described herein, means that the expandable balloon moves other bodily tissue into contact with the hemorrhage location. For example, indirect pressure may be applied to the hemorrhage location by inflating an expandable balloon in an abdominal cavity to move other intra-abdominal organ tissue such as the intestines, stomach, omentum, liver or spleen of a patent into contact with the hemorrhage location. In other embodiments, the hemorrhage control device may be used to stop other types of internal bleeding in other areas of the human or animal body.

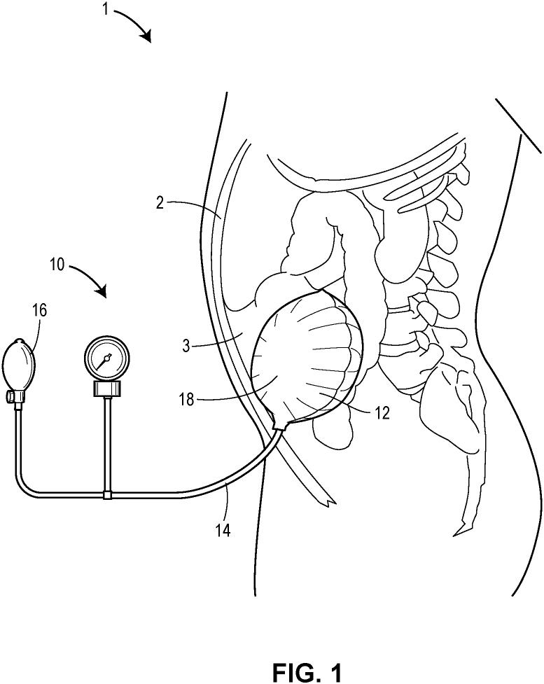

Turning now to FIG. 1, one embodiment of a hemorrhage control device 10 is illustrated inserted into a patient 1 through the patient’s abdominal wall 2.

The hemorrhage control device 10 includes a force-producing surface, such as an expandable balloon 12, and a positioning device in the form of an elongated member, such as a trocar 14. The trocar 14 positions the expandable balloon 12 through the abdominal wall 2 and into a peritoneal cavity 3, where a hemorrhage is located. The hemorrhage control device 10 further includes an inflation mechanism or source of pressurized fluid, such as a hand pump 16 that selectively inflates the expandable balloon 12. An outer surface 18 of the expandable balloon 12 applies pressure either directly to a hemorrhage location, or indirectly to the hemorrhage location by moving internal tissue, such as internal organs, which press against the hemorrhage location.

Once inserted in the peritoneal cavity 3, the expandable balloon 12 is pushed out of the trocar 14 by a deployment device, such as a plunger.

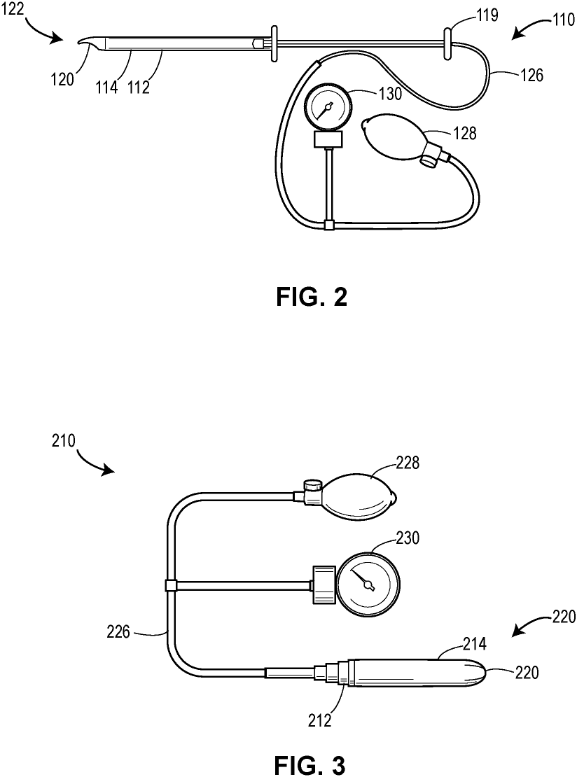

Turning now to FIG. 2, a first embodiment of a hemorrhage control device 110 is illustrated. The hemorrhage control device 110 includes a trocar 114 having an opening 120 at a distal end 122. In the illustrated embodiment, the distal end 122 may have a curved or hook-like shape to facilitate passage through the abdominal wall. In some embodiments, the curved distal end 122 may also be tapered. An expandable balloon 112 is disposed within the trocar 114, the expandable balloon 112 being collapsed within the trocar 114 in a stored condition, and the expandable balloon 112 being movable out of the trocar 114, through the opening 120, into a deployed condition, which is illustrated in FIG. 1. A deployment device, such as a plunger 119, may be slidably inserted into the trocar 114 to push the expandable balloon 112 out of the opening 120 and into the deployed condition. An inflation tube 126 is fluidly connected to an interior of the expandable balloon 112. A source of pressurized fluid, such as a hand pump 128, is connected to the inflation tube 126, pressurized fluid being introduced into the inflation tube 126 to pressurize and inflate the expandable balloon 112 in the deployed condition. In some embodiments, the source of pressurized fluid may include a heated and/or a cooled fluid. A pressure sensor, such as pressure gage 130, may be fluidly connected to the inflation tube 126 to measure pressure within the expandable balloon 112 in the deployed condition.

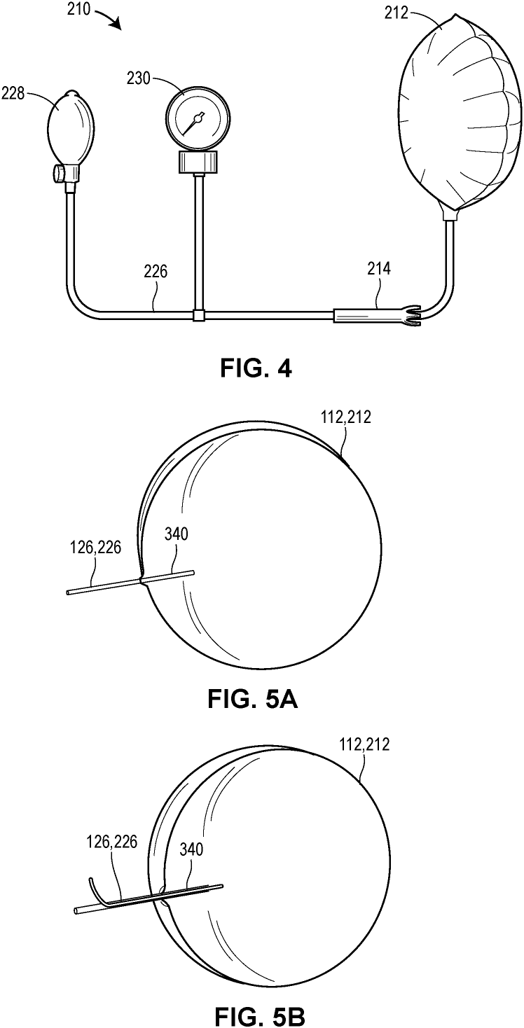

Turning now to FIGS. 3 and 4, a second embodiment of a hemorrhage control device 210 is illustrated. The hemorrhage control device 210 includes a trocar 214 having an opening 220 at a distal end 222. In the illustrated embodiment, the distal end 222 may have a plurality of flexible petals that shield the opening 220 during passage through the abdominal wall while deforming and separating to allow deployment of an expandable balloon 212. The expandable balloon 212 is disposed within the trocar 214, the expandable balloon 212 being collapsed within the trocar 214 in a stored condition, and the expandable balloon 212 being movable out of the trocar 214, through the opening 220, into a deployed condition, which is illustrated in FIG. 1. An inflation tube 226 is fluidly connected to an interior of the expandable balloon 212. A source of pressurized fluid, such as a hand pump 228, is connected to the inflation tube 226, pressurized fluid being introduced into the inflation tube 226 to pressurize and inflate the expandable balloon 212 in the deployed condition. A pressure sensor, such as pressure gage 230, may be fluidly connected to the inflation tube 226 to measure pressure within the expandable balloon 212 in the deployed condition.

Generally, the expandable balloon 112, 212 is capable of containing operating pressures from about 0 mm hg to about 200 mm hg. This is the normal operating pressure range of the expandable balloon 112, 212. However, in some embodiments, the expandable balloon 112, 212 may be capable of containing pressures above 200 mm hg. When inflated to the normal operating pressure of between about 0 mm hg and about 200 mm hg, the expandable balloon has an operating, fully inflated diameter of between about 5 cm and about 60 cm. The operating, fully inflated diameter, is measured when the expandable balloon 112, 212 is free to expand, and not constrained by other structures, such as body tissue. In other words, the fully inflated diameter is measured when the expandable balloon 112, 212 is outside of a body cavity. When inflated inside a body cavity, surrounding body tissue may constrain and prevent the expandable balloon 112, 212 from reaching its fully inflated diameter, although the expandable balloon 112, 212 in this case will advantageously apply direct pressure to the constraining body tissue, which advantageously results in the direct or indirect pressure being applied to the hemorrhage location. While a generally circular expandable balloon is illustrated, that inflates to a generally spherical shape, other embodiments may include different shapes for the expandable balloon. For example, in other embodiments, a generally square-shaped (uninflated) balloon may be used. In other embodiments, other shapes may be used to meet desired inflation characteristics or space requirements.

The expandable balloon 112, 212, may have an outer wall comprising a flexible material having a shore hardness of between about 70 A and 80D and a wall thickness of between about 0.001 and 0.003 in. In one embodiment, the outer wall of the expandable balloon 112, 212 may comprise a thermoplastic elastomer. In other embodiments, the expandable balloon 112, 212 may comprise other inflatable materials, such as, for example, polyester, nylon, polyurethane, Pebax®, silicone, and other thermoplastics or thermoplastic elastomers.

The trocar 114, 214 may have a first cross-sectional diameter, which is relatively small, in some embodiments between about 5 mm and about 15 mm, to allow insertion into a body cavity, thereby minimizing trauma to the surrounding tissue. The expandable balloon 112, 212 has a second cross-sectional diameter, generally in the range of between about 5 cm and about 60 cm, as set forth above when inflated to an operating pressure. The second cross-sectional diameter is measured when the expandable balloon is inflated to an operating pressure, and the second cross-sectional diameter is greater than 5 times larger than the first cross-sectional diameter.

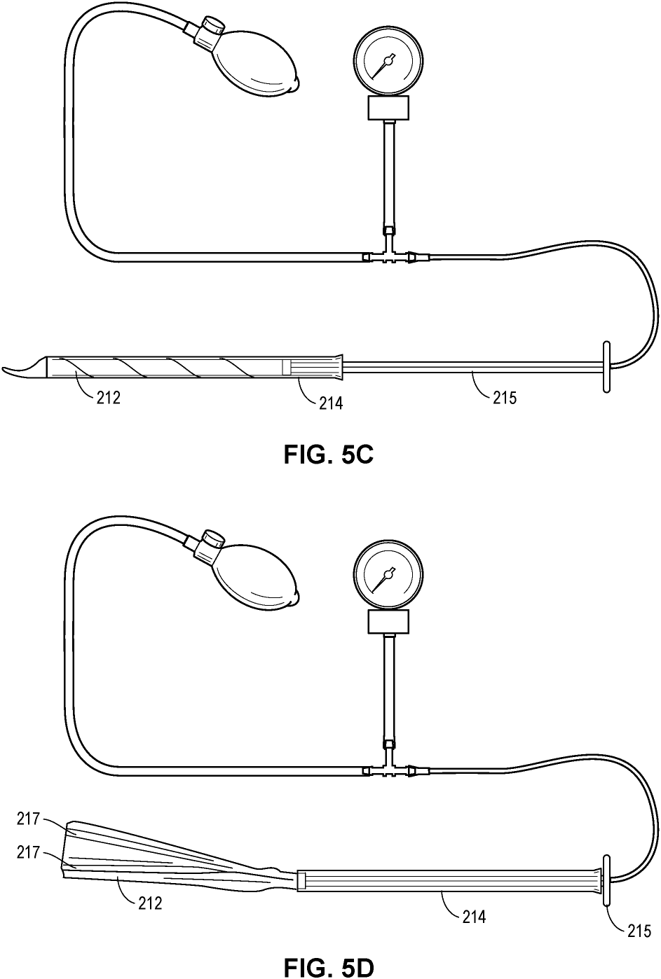

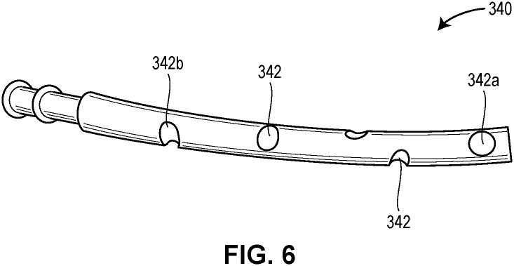

In either the first embodiment or the second embodiment, the inflation tube 126, 226 may be fluidly connected to an inflation baffle 340, as illustrated in FIGS. 5A, 5B, and 6. The inflation baffle 340 extends from a distal end of the inflation tube 126, 226 at least partially into the expandable balloon 112, 212. On some embodiments, the inflation baffle 340 may extend entirely across a diameter of the expandable balloon 112, 212 when the expandable balloon 112, 212 is inflated to an operational pressure. A length of the inflation baffle is advantageously greater than 20%, preferably greater than 50%, and more preferably greater than 90%, of the operating diameter of the expandable balloon 112, 212. This range reduces the chance of a hung or incomplete inflation of the expandable balloon 112, 212, when deployed in a body cavity.

The inflation baffle 340 includes a plurality of fluid openings 342 distributed along its length. For example, a first fluid opening 342a may be located near a distal end of the inflation baffle 340 and a second fluid opening 342b may be located at a proximal end of the inflation baffle 340. The fluid openings 342 may be distributed about the length of the inflation baffle 340 in random or organized fashion where at least two of the fluid openings are not axially aligned with one another. The fluid openings 342 may be organized to maximize the number of fluid openings 342 while not compromising the structural integrity of the inflation baffle 340. For example, in one embodiment, the fluid openings 342 may be separated from one another by at least 5 mm. The fluid openings 342 fluidly connect the inflation tube 126, 226, and thus the source of pressurized fluid, to the interior of the expandable balloon 112, 212. The inflation baffle 340 advantageously facilitates expansion of the expandable balloon 112, 212 by distributing the fluid pressure entering the interior of the expandable balloon 112, 212 across a large volume, which can expedite inflation even when the expandable balloon 112, 212 is kinked, pinched, or twisted.

In some embodiments, a physiological sensor may be connected to the expandable balloon or to the inflation tube. For example, a physiological sensor may be embedded in the outer wall of the expandable balloon, or the physiological sensor may be separate from the expandable balloon. The physiological sensor may be, for example, one of a pressure sensor, a heart rate monitor, a photplethysmograph, a pulse oximeter, and a thermometer. Readings from the physiological sensors may be used in a feedback mechanism to adjust internal pressure of the expandable balloon. In some cases, the physiological sensor may be located outside of the body cavity and connected to the feedback mechanism. For example, if a blood pressure sensor indicated that bleeding at the hemorrhage site had not occluded, a controller could instruct an automatic fluid pump to increase the internal pressure of the expandable balloon in an effort to fully occlude the hemorrhage. In other embodiments, an operator, such as a doctor, an EMT, or a nurse, may manually adjust the internal pressure of the expandable balloon based on the physiological sensor readings.

In some embodiments, fluid may be extracted from the body cavity through a lumen separate from the inflation tube or a lumen within the inflation tube. Fluid may be extracted to relieve pressure in the body cavity and/or to clean waste from the body cavity. In other embodiments, hemostatic or resuscitative adjuncts may be introduced into the peritoneal cavity through a lumen separate from an inflation tube or a lumen within the inflation tube.

In some embodiments, as illustrated in FIGS. 5C and 5D, the expandable balloon 212 is wrapped around an inflation baffle before introducing the expandable balloon 212 into a peritoneal cavity through the trocar 214. A deployment device, such as a plunger 215, may be used to push the expandable balloon 212 out of the trocar 214, as illustrated in FIG. 5D. Wrapping the expandable balloon 212 around the inflation baffle allows the expandable balloon 212 to deploy in a predictable manner when pressurized fluid is introduced into the expandable balloon 212. Another way of predictably storing the expandable balloon in the trocar is to fold the expandable balloon 212 into a plurality of pleats 217 around the inflation baffle before introducing the expandable balloon 212 into a peritoneal cavity through the trocar 214. In either case, the inflation baffle is located in a center of the balloon material when the balloon is in the stored condition.

A method of occluding non-compressible abdominal hemorrhage may include introducing the expandable balloon 112, 212 into a patient peritoneal cavity through the trocar 114, 214. The expandable balloon 112, 212 is moved out of the trocar 114, 214 and inflated in the peritoneal cavity of a patient by delivering pressurized fluid from the source of pressurized fluid 128, 228 into the expandable balloon 112, 212. Pressure is applied to a hemorrhage site by the expandable balloon 112, 212, as the expandable balloon 112, 212 inflates. In some embodiments, the trocar 114, 214 is inserted into the peritoneal cavity until the distal end 122, 222 of the trocar is in a desired location in the peritoneal cavity. The expandable balloon 112, 212, is pushed out of the opening 120, 220 in the trocar 114, 214 into a deployed condition outside of the trocar 114, 214 and inside the peritoneal cavity. Optionally, the trocar 114, 214 may be removed before inflating the expandable balloon 112, 212. The expandable balloon 112, 212, is then inflated to an internal pressure of at least 20 mm hg, which causes the outer wall of the expandable balloon 112, 212 to expand outward. In some embodiments, the internal pressure of the expandable balloon 112, 212, may be adjusted based on continued bleeding or blood pressure readings. To enhance occlusion of the hemorrhage location, an optional rigid support member may be used and external pressure may be applied with an external pressure device. The external pressure device may include a rigid back plate.

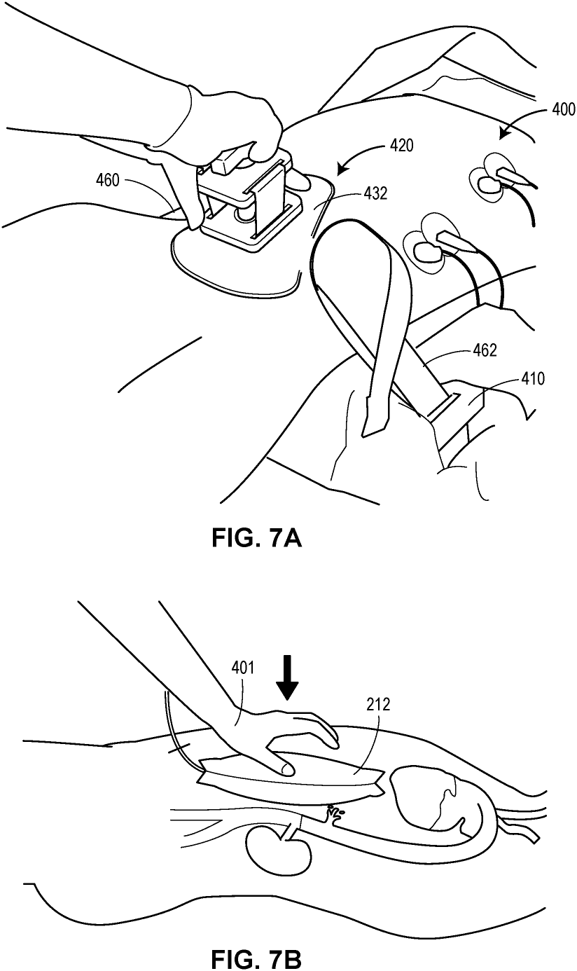

Turning now to FIGS. 7A and 7B, external pressure may be applied to an outer surface of a patient body by an external pressure device, such as a mechanical external pressure device 400 (FIG. 7A), or a human had 401 (FIG. 7B). Applying external pressure to a patient body may enhance or target pressure produced by the inflatable balloon 212 to occlude or reduce internal hemorrhage.

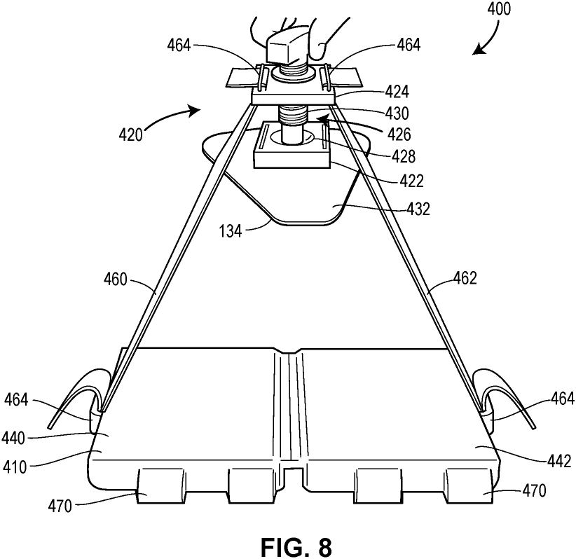

As illustrated in FIGS. 7A and 8, one embodiment of a mechanical external pressure device 400 may be used alone, or in conjunction with the hemorrhage control device described above, to stop hemorrhages in various locations. In other embodiments, an inflatable girdle may be used to supply external pressure.

The external pressure device 400, includes in one embodiment, a rigid platform 410 and a compression device 420 adjustably secured to the rigid platform 410. The compression device 420 includes a pressure plate carriage 422 and a strap carriage 424 that is linearly translatable relative to the pressure plate carriage 422. The pressure plate carriage 422 may also be considered to be linearly translatable relative to the strap carriage 424.

In some embodiments, the pressure plate carriage 422 and the strap carriage 424 are connected by a screw 426. More specifically, the pressure plate carriage 422 is connected to one end of the screw 426 so that when the screw 426 is rotated in a first direction, the pressure plate carriage 422 translates away from the strap carriage 424, and when the screw is rotated in a second direction, the pressure plate carriage translates towards the strap carriage 424. In some embodiments, the screw 426 comprises a first screw 428 nested within a second screw 430.

A pressure plate 432 is removably attached to the pressure plate carriage 422. The pressure plate 432 may have various shapes. For example, the pressure plate 432 may have any one of a generally triangular shape, a generally rectangular shape, a generally circular shape, a generally oval shape, or any combination thereof. A bottom surface of the pressure plate 432 may be shaped to fit a certain portion of the human body. For example, the bottom surface of the pressure plate 432 may be convex shaped, for example to compliment the groin area, the bottom surface of the pressure plate 432 may be concave shaped, for example to compliment a side of the leg or arm, or the bottom surface of the pressure plate 432 may be angled with respect to the pressure plate carriage 422, for example to compliment a side of the torso. In yet other embodiments, the bottom surface of the pressure plate 432 may combine any of the aforementioned shapes to better complement a location on the body having a hemorrhage. In some embodiments, the bottom surface of the pressure plate may include an absorbent layer 434 and/or may include a clotting agent to enhance blood clotting. By form fitting the bottom surface of the pressure plate 432 to the hemorrhage location, the pressure plate 432 can slow or stop bleeding more quickly, for example, by providing targeted pressure at the hemorrhage location. Additionally, the removable attachment of the pressure plate 432 to the pressure plate carriage 422 makes changing of the pressure plate 432 quick and easy. The pressure plate 432 may be removably attached to the pressure plate carriage 422 by any removable connection that provides stability to the pressure plate 432 when connected while allowing quick and easy removal from the pressure plate carriage 422. Some example removable connections include, but are not limited to, a snap-fit connection, a magnetic connection, and a removable fastener connection. A plurality of pressure plates 432 may be included in a kit with the external pressure device 400, each pressure plate 432 having a different shape, to give a user many options for selecting an optimal shape for a particular bleeding location. In some embodiments, the pressure plate 432 may include movable sections that allow the overall shape of the pressure plate 432 to be changed to better fit a bleeding location.

The rigid platform 410 comprises a first rigid housing 440 and a second rigid housing 442 that are pivotably connected to one another, for example by a hinge. The pressure plate 432, the screw 426, the pressure plate carriage 424 and a plurality of straps 460, 462, may be stored within an internal pocket between the first rigid housing 440 and the second rigid housing 442 when the first and second rigid housings 440, 442 are in a closed configuration (not shown).

In an open and deployed position (FIG. 8), the first rigid housing 440 and the second rigid housing 442 form a stable platform for a portion of a body having a hemorrhage. In a closed position (not shown), the first rigid housing 440 and the second rigid housing 442 form an enclosed container that protects components and devices that may be used for hemorrhage control. The enclosed container is compact and easy to carry as well as rugged and damage resistant and waterproof.

The compression device 420 is adjustably secured to the rigid platform 410 with the first adjustable strap 460 and the second adjustable strap 462. The first adjustable strap 460 is connected to the first rigid housing 440 and the second adjustable strap 462 is connected to the second rigid housing 442. The first adjustable strap 460 and the second adjustable strap 462 facilitate gross or large adjustments in the compression device 420 during initial placement of the compression device 420. The first adjustable strap 460 and the second adjustable strap 462 may include adjustment loops 464 located on the rigid platform 410 and on the strap carriage 424.

A plurality of interlocking teeth 470 may be formed on the outer side surfaces of the first rigid housing 440 and on the second rigid housing 442. The interlocking teeth 470 form stabilizing buttresses with the ground when the first rigid housing 440 and the second rigid housing 442 are in the open and deployed position (FIG. 8), and the interlocking teeth 470 align the first rigid housing 440 and the second rigid housing 442 when moving towards a closed position by interlocking with one another.

The disclosed hemorrhage control device and external pressure device provide hemorrhage control for the management of trauma and a reduction of blood flow below the diaphragm to reduce intraperitoneal hemorrhage and to enhance coronary and cerebral perfusion. Studies have shown that, although over half of the tissue beds are below the diaphragm, approximately two-thirds of bleeding that leads to hemorrhagic shock occurs below the diaphragm. Therefore, the ability to control bleeding below the diaphragm, and especially in the peritoneal cavity, provides a significant advantage particularly in management of trauma. This is particularly useful in treating patients who have suffered abdominal injuries from knives and guns, blunt trauma from falls, explosions, motor vehicle accidents, complications due to the delivery of babies from subdiaphragmatic hemorrhaging and other vascular catastrophes below the diaphragm such as ruptured abdominal aortic aneurysms. The disclosed hemorrhage control device and external pressure device are particularly useful in battlefield applications in which it is essential to be able to rapidly control life-threatening hemorrhage in a minimally invasive manner in order to avoid immediate death and complications from infections and the like until definitive repair of injuries can take place. Additionally, the ability to perform this procedure rapidly and effectively reduces the exposure of the medical personnel to battlefield injuries.

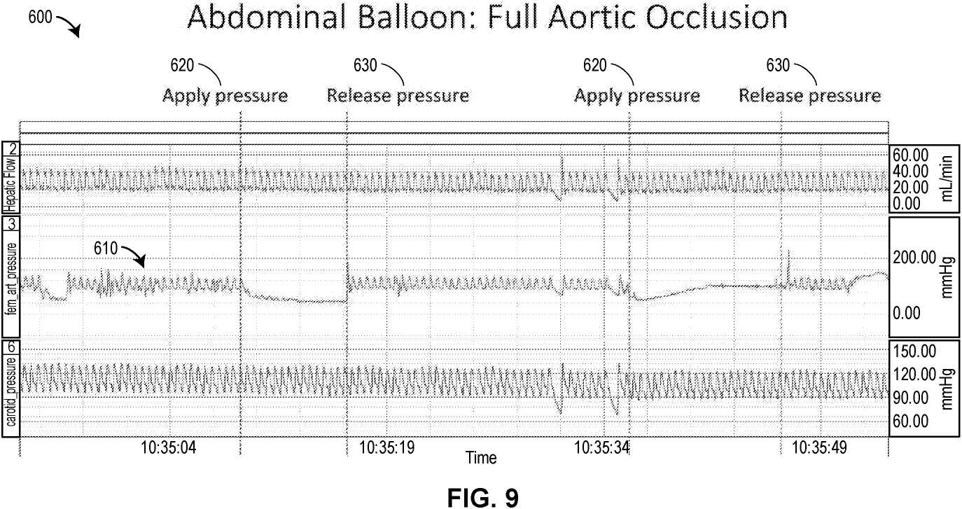

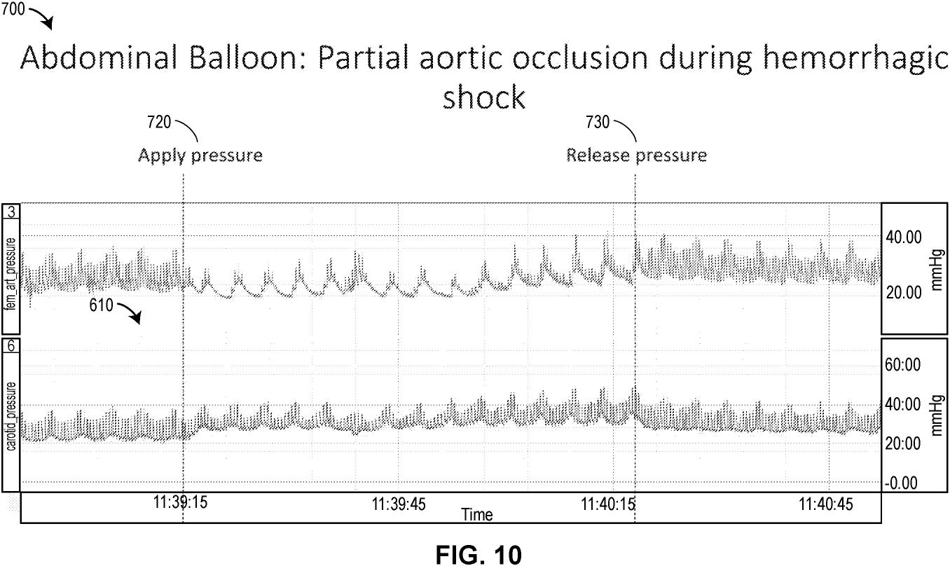

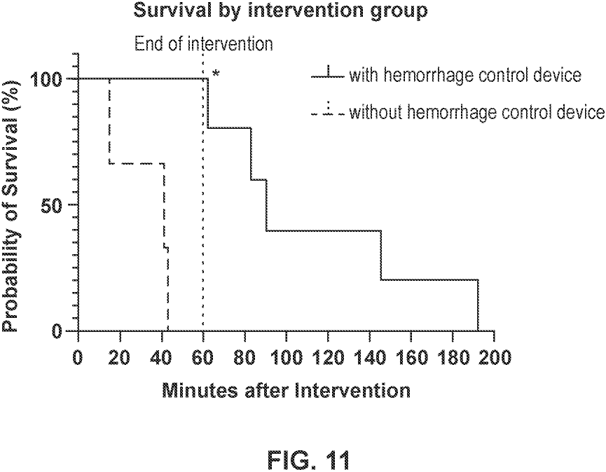

Turning now to FIGS. 9 and 10, the effectiveness of the hemorrhage control devices described herein is illustrated in tests involving full and partial aortic occlusion. FIG. 9 is a graph 600 of femoral artery blood pressure 610 below the level of the expandable balloon when the expandable balloon is inflated at 620 and deflated at 630. The uppermost tracing in FIG. 9 is one of hepatic artery blood flow and the bottom tracing is one of carotid artery pressure. As can be noted, hepatic artery blood flow is preserved even as full occlusion of the aorta is produced as evidenced by loss of the femoral artery pressure waveform. During this time it can be noted that carotid artery pressure is also preserved.

Similarly, FIG. 10 is a graph 700 of aortic blood pressure 710 (sampled from the femoral artery) as well as carotid artery pressure when the expandable balloon is inflated at 720 and deflated at 730. As can be noted, it is possible to provide partial occlusion as opposed to full occlusion of the aorta. The ability to preserve hepatic blood flow and the option to provide partial as opposed to full occlusion has significant physiologic advantages by providing an opportunity to reduce complete ischemia to vital organs compressed by the expandable balloon, such as the liver, while also providing hemostasis. This is advantageous over other techniques, such as resuscitative endovascular balloon occlusion of the aorta (REBOA), which generally produces complete ischemia of organ systems below the occlusion.

### Experimental Trial Results

The disclosed hemorrhage control device was tested on animals that were instrumented under general anesthesia for monitoring of hemodynamics and blood sampling. The specific test parameters may be found in McCracken, Brendan M., et al. “Novel intra-peritoneal hemostasis device prolongs survival in a swine model of non-compressible abdominal hemorrhage.” The Journal of Trauma and Acute Care Surgery (2021). Jan. 25, 2021 - Volume Publish Ahead of Print - doi: 10.1097/TA.0000000000003091, which is hereby incorporated by reference herein. The animals each exhibited a 30% controlled arterial hemorrhage followed by combinations of liver, spleen, and kidney injuries. In the subject group of animals, the hemorrhage control device disclosed herein was inflated and maintained for within the peritoneal cavity and maintained pressure for 60 minutes.

The results of the animals managed with hemorrhage control device were overwhelmingly positive. All of the animals treated with the hemorrhage control device survived the duration of the intervention period (60 minutes) while all control animals died at a time range of 15-43 minutes following the organ injury. Animals treated with the hemorrhage control device remained hemodynamically stable and experienced increased cardiac output and decreased shock index after inflation of the inflatable balloon.

Turning now to FIG. 11, a graph of survival probability vs time for the treated animals and the control animals is illustrated. While an increase in survivability in the animals treated with the hemorrhage control device was expected, the magnitude of the increase was unexpected. Animals treated with the hemorrhage control device had an increased survivability time of between about 70% and about 200% for the first hour after injury, which is the most critical hour in preventing brain damage from blood loss. Furthermore, because the test was terminated at 60 minutes, survivability is likely even longer for treated subjects.

Test results show that the disclosed hemorrhage control device is capable of prolonging survival by temporarily stanching lethal NTCH of the abdomen. The disclosed hemorrhage control device is an effective temporary countermeasure to NCTH of the abdomen that could be deployed in the pre-hospital environment or as a bridge to more advanced therapy.

The hemorrhage control device disclosed herein has been shown to prolong survival in normally highly lethal multi-organ traumatic hemorrhage, which indicates that it is capable of serving as an effective bridge to definitive therapeutic hemostasis. In addition to prolonging survival, the disclosed hemorrhage control device slows hemodynamic deterioration. While not being bound by theory, the slowing of hemodynamic deterioration is understood to be an effect of indirect tamponade as the balloon cannot directly contact all the bleeding surfaces since they occur at different levels in the peritoneal cavity. The balloon inflation within the closed peritoneal space leads to displacement of adjacent abdominal tissues which then contact the injury sites and provide pressure, slowing the hemorrhage. As a result, the disclosed hemorrhage control device preserves overall physiologic reserve which could result in a more effective resuscitation subsequent to the intervention and definitive surgical hemostasis.

The disclosed hemorrhage control device is advantageously versatile. The disclosed hemorrhage control device is capable of hemorrhage control without necessarily requiring occlusion of the aorta. The disclosed hemorrhage control device, when activated, does not necessarily stop hepatic artery flow. Despite not stopping hepatic artery flow, the disclosed hemorrhage control device was able to control the severe hemorrhage occurring from the hepatic injury, indicating that it is effective in high zone two injuries where use of other known tools would not be effective. Alternatively, complete aortic occlusion can be selectively produced using the disclosed hemorrhage control device with manual compression of the abdomen when the balloon is inflated. Testing shows that when the balloon is inflated, aortic impingement occurs inferior to the celiac trunk, which indicates it may also be useful for low-abdominal organ or deep pelvic vascular injury.

The potential to quickly deploy the disclosed hemorrhage control device for short-term stanching of multiple abdominal organ hemorrhage, combined with user selective application of distal aortic occlusion if needed, and easy removal, advantageously produce a flexible solution to the current limitations of approved aortic occlusion techniques, or other invasive hemostatic technologies. The ability to control inflation pressures as well as to add additional external pressure may also allow a more titrated approach. The ability to control hemorrhage without the need for total aortic occlusion may also be beneficial in reducing the level of acquired oxygen debt and the severity of subsequent reperfusion injury. Lastly, use of the disclosed hemorrhage control device will not commit patients to surgery if nonsurgical hemostasis techniques such as interventional radiology can be used to obtain hemostasis.

Changes and modifications in the specifically described embodiments can be carried out without departing from the principles of the invention. For example, electrodes can be applied to the balloons for use in cardiac pacing and defibrillation if deployed in the chest cavity. Although balloons and cuffs may be inflated using air, other techniques involving hydraulic fluids and mechanical actuators may suggest themselves to those skilled in the art. Although inflatable devices are illustrated as spherical or annular, other shapes could be used such as cylindrical, pill-shaped, and the like. Also, the various elements of each illustrated embodiment of the invention can be combined and substituted with other of the embodiments. The embodiments are provided in order to illustrate the invention and should not be considered limiting. The described methods and devices are to be limited only by the scope of the appended claims.

1. A hemorrhage control device, comprising:
a trocar having an opening at a distal end;
an expandable balloon disposed within the trocar, the expandable balloon being collapsed within the trocar in a stored condition, and the expandable balloon being movable out of the trocar, through the opening, into a deployed condition;
an inflation tube fluidly connected to an interior of the expandable balloon; and
a source of pressurized fluid connected to the inflation tube, pressurized fluid being introduced into the inflation tube to pressurize and inflate the expandable balloon in the deployed condition.

2. (canceled)

3. The hemorrhage control device of claim 1, wherein the expandable balloon is capable of containing internal pressures from about 0 mm hg to about 200 mm hg.

4. The hemorrhage control device of claim 1, wherein the expandable balloon has an external diameter of between about 5 cm and about 60 cm when internal pressure of the expandable balloon is at an operating pressure and not externally constrained.

5. The hemorrhage control device of claim 1, wherein the trocar has a first cross-sectional diameter and the expandable balloon has a second cross-sectional diameter, the second cross-sectional diameter being measured when the expandable balloon is inflated to an operating pressure, and the second cross-sectional diameter capable of being greater than 5 times larger than the first cross-sectional diameter.

6. The hemorrhage control device of claim 1, further comprising an inflation baffle within the expandable balloon, the inflation baffle being fluidly connected to the inflation tube.

7. The hemorrhage control device of claim 6, wherein the inflation baffle includes a plurality of fluid openings distributed along a length of the inflation baffle.

8. The hemorrhage control device of claim 6, wherein a length of the inflation baffle is greater than 20%, preferably greater than 50%, and more preferably greater than 90%, of a diameter of the expandable balloon, when the expandable balloon is inflated to an operating diameter.

9-10. (canceled)

11. The hemorrhage control device of claim 1, wherein a distal end of the trocar comprises a deformable tip.

12. The hemorrhage control device of claim 1, wherein a distal end of the trocar comprises a curved tip.

13. (canceled)

14. The hemorrhage control device of claim 1, further comprising a rigid external support member.

15. (canceled)

16. The hemorrhage control device of claim 1, further comprising a physiological sensor connected to the expandable balloon or to the inflation tube.

17-21. (canceled)

22. The hemorrhage control device of claim 7, wherein at least two openings in the plurality of fluid openings are not axially aligned with one another.

23. A method of occluding non-compressible abdominal hemorrhage, the method comprising:
introducing an expandable balloon into a peritoneal cavity through a trocar;
moving the expandable balloon out of the trocar;
inflating the expandable balloon in the peritoneal cavity; and
applying pressure to a hemorrhage site by the expandable balloon.

24. The method of claim 23, wherein applying pressure to the hemorrhage site includes applying indirect pressure by moving patient tissue to occlude the hemorrhage site.

25. (canceled)

26. The method of claim 23, wherein inflating the expandable balloon includes inflating the expandable balloon to an internal pressure of at least 20 mm hg.

27. The method of claim 23, further comprising adjusting internal balloon pressure based on continued bleeding or blood pressure readings.

28. The method of claim 23, further comprising applying pressure external to the peritoneal cavity.

29. (canceled)

30. The method of claim 23, further comprising extracting fluid from the peritoneal cavity through a lumen separate from an inflation tube or a lumen within the inflation tube.

31. The method of claim 23, further comprising introducing hemostatic, antibacterial, or resuscitative adjuncts into the peritoneal cavity through a lumen separate from an inflation tube or a lumen within the inflation tube.

32-35. (canceled)

36. The method of claim 23, further comprising folding the expandable balloon into a plurality of pleats around an inflation baffle before introducing the expandable balloon into a peritoneal cavity through the trocar.
